(19) United States
(12) Patent Application Publication
(10) Pub. No.: US 2023/0404590 A1
(43) Pub. Date: Dec. 21, 2023

(54) INVERTING BRAIDED ANEURYSM TREATMENT SYSTEM HAVING A SEMI-FRUSTOCONICALLY-SHAPED PORTION

(71) Applicant: DePuy Synthes Products, Inc., Raynham, MA (US)

(72) Inventors: Ruijiao XU, Miami Lakes, FL (US); Robert SLAZAS, Raynham, MA (US); Lacey GOROCHOW, Miami, FL (US)

(73) Assignee: DePuy Synthes Products, Inc., Raynham, MA (US)

(21) Appl. No.: 17/840,906

(22) Filed: Jun. 15, 2022

(51) Int. Cl. A61B 17/12

(52) U.S. Cl. CPC A61B 17/12172; A61B 17/12113; A61B 2017/1205; A61B 2017/00292

(57) ABSTRACT

The disclosed technology generally relates to braided implants for aneurysm therapy. The disclosed technology can include a system having a tubular braid comprising an open end, a pinched end, and a predetermined shape. In the predetermined shape, the tubular braid can include a first segment extending from the open end to a first inversion, a second segment extending from the first inversion to a second inversion and forming a bag comprising a semi-frustoconically-shaped portion proximate the first inversion such that the semi-frustoconically-shaped portion defines an inner channel forming an opening to the bag, and a third segment surrounded by the second segment and extending from the second inversion to the pinched end. The system can include a catheter having a lumen therethrough, a distal end, and an outer diameter at the distal end being sized to be inserted into the bag through the opening of the bag.

### FIELD OF INVENTION

The present invention generally relates to medical instruments, and more particularly, to braided implants for aneurysm therapy.

### BACKGROUND

Cranial aneurysms can be complicated and difficult to treat due to their proximity to critical brain tissues. Prior solutions have included endovascular treatment whereby an internal volume of the aneurysm sac is removed or excluded from arterial blood pressure and flow. Current alternatives to endovascular or other surgical approaches can include intravascularly delivered treatment devices that fill the sac of the aneurysm with embolic material or block the entrance or neck of the aneurysm. Both approaches attempt to prevent blood flow into the aneurysm. When filling an aneurysm sac, the embolic material clots the blood, creating a thrombotic mass within the aneurysm. When treating the aneurysm neck, blood flow into the entrance of the aneurysm is inhibited, inducing venous stasis in the aneurysm, and facilitating a natural formation of a thrombotic mass within the aneurysm.

Current intravascularly delivered devices typically utilize embolic coils or tubular braided implants to either fill the sac or treat the entrance of the aneurysm. Naturally formed thrombotic masses formed by treating the entrance of the aneurysm can result in improved healing compared to aneurysm masses packed with embolic coils because naturally formed thrombotic masses can reduce the likelihood of distention from arterial walls and facilitate reintegration into the original parent vessel shape along the neck plane. Embolic coils delivered to the neck of the aneurysm, however, can potentially have the adverse effect of impeding the flow of blood in the adjoining blood vessel, particularly if the entrance is overpacked. Conversely, if the entrance is insufficiently packed, blood flow can persist into the aneurysm.

Tubular braided implants, on the other hand, eliminate many of the problems of using embolic coils but can be difficult to install into the aneurysm properly. For example, tubular braided implants can become twisted when installing the braided implant into an aneurysm, requiring removal of the braided implant or difficult maneuvering to de-twist the braided implant. To illustrate, FIG. 1A depicts an existing braided aneurysm implant 100 in a deployed configuration. As illustrated in FIG. 1A, in the deployed configuration, the implant 100 can have an inner braid 105, an outer braid 115, and an inner channel 127 formed between the inner braid 105 and the outer braid 115. When the implant 100 is transitioned to the deployed configuration, the implant 100 can become twisted at a twist point 102 at the inner channel 127 (as illustrated in FIG. 1B) preventing installation of the implant 100. The implant 100 must then be removed or maneuvered to de-twist the implant 100.

To help explain how an existing implant 100 can become twisted when being installed, FIGS. 2A-2H depict an existing implant 100 having a braid 110 being expanded to a predetermined shape as the braid 110 exits a lumen extending through a microcatheter 160. The implant 100 has a predetermined shape similar to that illustrated in FIG. 1A. As illustrated in FIG. 2A, the braid 110 is shaped to a delivery shape that is extended to a single layer of tubular braid having a compressed circumference/diameter sized to be delivered through the microcatheter 160 and a length L. During delivery through the microcatheter 160, a detachment feature 150 can be attached to a delivery system at a proximal end of the implant 100, a pinched end 112 can be positioned near the proximal end of the implant 100, and an open end 114 can define the distal end of the implant 100.

As illustrated in FIG. 2B, the open end 114 can be positioned to exit the microcatheter 160 before any other portion of the braid 110 exits the microcatheter 160. The open end 114 can expand as it exits the microcatheter 160.

As illustrated in FIG. 2C, the distal portion of the braid 110 can continue to expand radially as it exits the microcatheter 160. It is generally at this stage where existing implants 100 can become twisted at a twist point 102 (as illustrated in FIG. 1B) that can form as the implant 200 exits the microcatheter 160. The twist point 102 can generally align with the inner channel 127 depicted in FIG. 1A.

The remaining figures (FIGS. 2D-2H) illustrate the implant 100 as if it had expanded properly and had not become twisted. As illustrated in FIG. 2D, the braid 110 can form the inversion 122 defining the outer segment 142 as the braid 110 is further pushed out of the microcatheter 160. As illustrated in FIGS. 2E through 2G, the “S” shape of the middle segment 144 can begin to form as the braid 110 is further pushed from the microcatheter 160.

As illustrated in FIG. 2H, when all, or nearly all of the braid 110 exits the microcatheter 160, the braid 110, not confined by an aneurysm, can expand to a predetermined shape similar to the shape illustrated in FIG. 1A. As will be appreciated, if the implant 100 becomes twisted at the twist point 102, the implant 100 will be unable to transition to the predetermined shape properly and will either need to be removed or de-twisted.

What is needed, therefore, is a tubular braided implant that is configured to reduce the likelihood that the tubular braided implant will become twisted during installation and to increase the effectiveness of the braided implant. These and other problems can be addressed by the disclosed technology.

### SUMMARY

It is an object of the present designs to provide devices and methods to meet the above-stated needs. Generally, it is an object of the present invention to provide a system having a tubular braid comprising an open end, a pinched end, and a predetermined shape. In the predetermined shape, the tubular braid can include a first segment extending from the open end to a first inversion, a second segment extending from the first inversion to a second inversion and forming a bag comprising a semi-frustoconically-shaped portion proximate the first inversion such that the semi-frustoconically-shaped portion defines an inner channel forming an opening to the bag, and a third segment surrounded by the second segment and extending from the second inversion to the pinched end. The system can include a catheter having a lumen therethrough, a distal end, and an outer diameter at the distal end being sized to be inserted into the bag through the opening of the bag.

The semi-frustoconically-shaped portion can be configured to prevent the tubular braid from becoming twisted proximate the inner channel when deployed.

The semi-frustoconically-shaped portion can be configured such that a first distance between an apex of the semi-frustoconically-shaped portion and the first segment is greater than a second distance between a trough of the semi-frustoconically-shaped portion and the first segment. The apex can be nearer the inner channel than the trough. The first distance can be approximately 1 millimeter and the second distance can be approximately 0.37 millimeters.

The semi-frustoconically-shaped portion can form an indentation into the bag proximate the opening.

The inner channel can extend from the first inversion to the apex of the semi-frustoconically-shaped portion.

The tubular braid can be stable in an implanted shape based on the predetermined shape when constricted by a substantially spherical cavity. In the implanted shape, at least a portion of the first segment can be positioned to contact a cavity wall of the substantially spherical cavity. In the implanted shape, a proximal inversion corresponding to the first inversion of the predetermined shape can be positioned at an entrance to the substantially spherical cavity and the bag can be positioned within the substantially spherical cavity.

In the implanted shape, the opening of the bag can be accessible at the entrance to the substantially spherical cavity and the opening can be configured to receive the distal end of the catheter into the bag.

In the implanted shape, the opening can be resilient to expand to receive the distal end of the catheter and contract when the catheter is removed from the opening.

In the implanted shape, the proximal inversion can be configured such that the first segment forms an approximately flat surface proximate the entrance to the substantially spherical cavity.

In the predetermined shape, the tubular braid can be cylindrically symmetrical about a central axis and the inner channel can extend in a proximal direction from the bag, constrict about the central axis, and define the opening of the bag.

A diameter of the inner channel when the braid is in the predetermined shaped can collapse when the braid is in the implanted shape.

The inner channel can be sized to facilitate clotting of blood when the braid is in the implanted shape.

An outer profile of the tubular braid in the predetermined shape can be approximately a right cylinder. In the predetermined shape, the open end can encircle the bag.

The tubular braid can include a shape memory material configured to self-expand into the predetermined shape. The tubular braid can include at least one of nitinol and platinum.

The disclosed technology can include tubular braid having an open end, a pinched end, and a predetermined shape. The predetermined shape can include a first segment that extends from the open end to a first inversion, second segment that extends from the first inversion to a second inversion and forming a bag comprising a semi-frustoconically-shaped portion proximate the first inversion such that the semi-frustoconically-shaped portion defines an inner channel forming an opening to the bag. The predetermined shape can include a third segment that is surrounded by the second segment and extends from the second inversion to the pinched end.

The semi-frustoconically-shaped portion can be configured to prevent the tubular braid from becoming twisted proximate the inner channel when deployed.

The semi-frustoconically-shaped portion can be configured such that a first distance between an apex of the semi-frustoconically-shaped portion and the first segment is greater than a second distance between a trough of the semi-frustoconically-shaped portion and the first segment. The apex can be nearer the inner channel than the trough. The first distance can be approximately 1 millimeter and the second distance can be approximately 0.37 millimeters.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1A is an illustration of an existing implant having a tubular braid in a predetermined shape;

FIG. 1B is an image of a twist formed in an existing implant having a tubular braid;

FIGS. 2A through 2H are illustrations of an existing implant having a tubular braid that expands to a predetermined shape as the tubular braid exits a microcatheter;

FIG. 3 is an illustration of an implant having a tubular braid in a predetermined shape with a semi-frustoconically-shaped portion, in accordance with aspects of the disclosed technology;

FIG. 4 is an illustration of an implant having a tubular braid that expands to a predetermined shape with a semi-frustoconically-shaped portion as the tubular braid exits a microcatheter, in accordance with aspects of the disclosed technology;

FIG. 5 is an illustration of the implant having the tubular braid with the semi-frustoconically-shaped portion in an implanted shape in an aneurysm;

FIGS. 6-8 are illustrations of various example implants having a tubular braid in a predetermined shape with a semi-frustoconically-shaped portion in accordance with aspects of the disclosed technology.

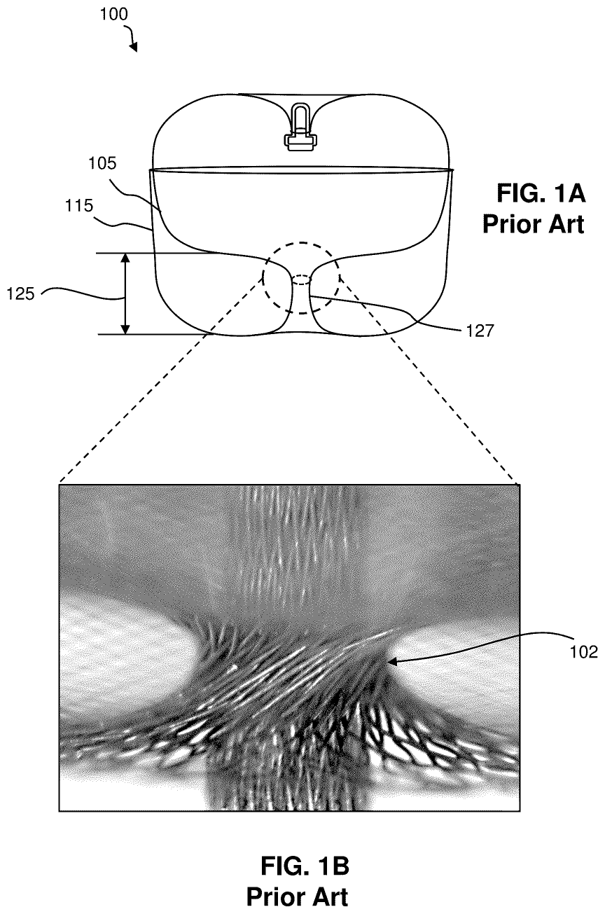

### DETAILED DESCRIPTION

The examples of the disclosed technology described herein address many of the deficiencies associated with traditional braided implants including the tendency of the braided implant to twist during insertion, preventing proper deployment of the braided implant. Furthermore, the examples of the disclosed technology can facilitate effective clotting at the aneurysm by reducing a gap between the inner braid and the outer braid and thus providing a greater amount of material proximate an opening of the aneurysm. As will become apparent, reducing the gap between the inner braid and the outer braid can promote blood stasis to facilitate blood flow diversion and aneurysm healing.

Examples presented herein generally include a braided implant that can be secured within an aneurysm sac and occlude a majority of the aneurysm's neck. The implant can include a tubular braid that can be set into a predetermined shape, compressed for delivery through a microcatheter, and implanted in at least one implanted position that is based on the predetermined shape and the geometry of the aneurysm in which the braid is implanted. The predetermined shape can include a semi-frustoconically-shaped portion that can help reduce the likelihood that the braided implant can become twisted when transitioning to a deployed configuration and can reduce a gap between the inner braid and the outer braid when in the deployed configuration. The disclosed technology is not necessarily limited to the examples described, which can be varied in construction and detail.

The terms “distal” and “proximal” are used throughout the preceding description and are meant to refer to a positions and directions relative to a treating physician. As such, “distal” or “distally” refer to a position distant to or a direction away from the physician. Similarly, “proximal” or “proximally” refer to a position near or a direction toward the physician. Furthermore, the singular forms “a,” “an,” and “the” include plural referents unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As used herein, the terms “semi-frustoconical” or “semi-frustoconically-shaped” can refer to a shape being generally frustoconical. In other words, the terms “semi-frustoconical” or “semi-frustoconically-shaped” can refer to a shape similar to the frustrum of a cone. Furthermore, the terms “semi-frustoconical” or “semi-frustoconically-shaped” can refer to components, whether solid or hollow, that include or define features such as a channel extending therethrough and sharp or rounded edges. Further features and benefits of the disclosed technology will become apparent throughout this disclosure.

As used throughout this disclosure, the term “deployed configuration” can refer to a configuration of a braided implant when fully deployed, whether or not the braided implant is installed in an aneurysm sac.

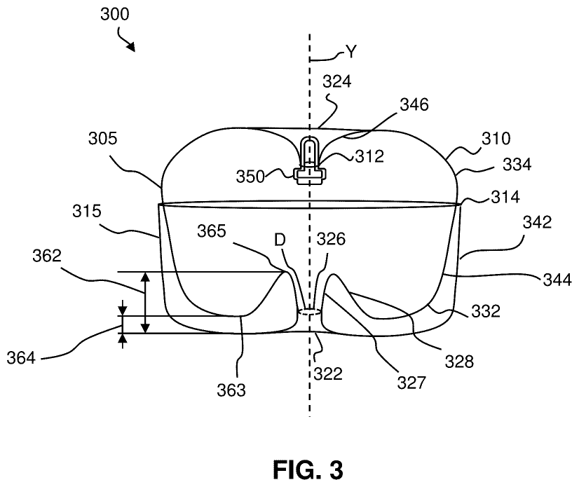

Turning now to the Figures in which like numerals represent like elements, FIG. 3 illustrates an example braided implant 300 of the disclosed technology that can have a predetermined shape with a semi-frustoconically-shaped portion 328. As will become apparent throughout this disclosure, the implant 300 can be configured to reduce the likelihood that the implant 300 will become twisted when transitioning to the deployed configuration (as illustrated in FIG. 3). Furthermore, as will be appreciated by one of skill in the art, the implant 300 can be configured to treat a range of aneurysm sizes, including large and small aneurysms.

The implant 300 can include a tubular braid 310 having an open end 314 and a pinched end 312. The implant 300 can include a detachment feature 350 attached to the tubular braid 310 at the pinched end 312. The tubular braid 310 can be formed in the predetermined shape (FIG. 3), collapsed for delivery through a microcatheter (i.e., microcatheter 160), attached to a delivery system at the detachment feature 350, and implanted in a shape similar to that depicted in FIG. 3.

When in the predetermined shape, the tubular braid 310 can include two inversions 322, 324, dividing the braid 310 into three segments 342, 344, 346. The first segment 342 can form the outer braid 315 while the second and third segments 344, 346 can form the inner braid 305. In the predetermined shape, the first segment 342 can extend from the open end 314 of the braid 310 to the first inversion 322 (a “proximal inversion”), a third segment 346 extending from the pinched end 312 of the braid 310 to the second inversion 324 (a “distal inversion”), and a second segment 344 extending between the two inversions 322, 324 and forming a bag. When in the predetermined shape, the tubular braid 310 can be substantially radially symmetrical about a central vertical axis Y and form a generally right cylinder and the open end 314 can encircle the bag. FIG. 3 illustrates a profile of each segment 342, 344, 346, and the detachment feature 350 is illustrated as a flat key that can be used with a mechanical implant delivery system (not illustrated).

The second segment 344 can have one or more bends 332, 334 to help form the bag. The bends 332, 334 can be positioned to facilitate the movement of the braid 310 into the deployed configuration illustrated in FIG. 3 and the bends 332, 334 can be positioned to stabilize the braid 310 in the deployed configuration.

As non-limiting examples, the braid 310 of the illustrated implant can have a diameter between about 1 mm and about 20 mm and a height between about 0.5 mm and about 25 mm when in the predetermined shape. In other examples, the braid 310 of the illustrated implant can have a diameter between about 3 mm and about 10 mm and a height between about 4 mm and about 20 mm when in the predetermined shape. In yet other examples, the braid 310 of the illustrated implant can have a diameter between about 6 mm and about 6.5 mm and a height between about 5 mm and about 5.5 mm when in the predetermined shape Furthermore, the length of the braid 310 in the delivery shape can be greater than the diameter of the braid 310 when in the predetermined shape. As a non-limiting example, the ratio of the outermost diameter of the braid 310 in the predetermined shape to the length of the braid 310 in the delivery shape can be between about 0.5 and about 0.2. As another non-limiting example, the ratio of the outermost diameter of the braid 310 in the predetermined shape to the length of the braid 310 in the delivery shape can be between about 0.3 and about 0.24.

The tubular braid 310 can include a memory shape material that can be heat set to the predetermined shape, can be deformed for delivery through a catheter, and can self-expand to an implanted shape that is based on the predetermined shape and confined by the anatomy of the aneurysm in which it is implanted. The memory shape material can be or include nickel, titanium, nickel and titanium (i.e., nitinol), platinum, cobalt-chrome, stainless steel, alloys of any of the foregoing, and/or other suitable biocompatible materials for the application.

As illustrated in FIG. 3, the implant 300 can include a semi-frustoconically-shaped portion 328 that can be part of the second segment 344 (part of the bag formed by the second segment 344). The semi-frustoconically-shaped portion 328 can be disposed proximate the first inversion 322 and can define an inner channel 327 that can form an opening 326 to the bag. The inner channel 327 can extend along the central vertical axis Y from the opening first inversion 322 to an apex 365 of the semi-frustoconically-shaped portion 328. Stated otherwise, the semi-frustoconically-shaped portion 328 can indent into the second segment 344 to form a somewhat volcano-like structure in the second segment 344, forming the inner channel 327 and a bottom portion of the second segment 344.

As illustrated in FIG. 3, the apex 365 of the semi-frustoconically-shaped portion 328 can be a portion of the semi-frustoconically-shaped portion 328 that is disposed farther away from the second segment 344 than a trough 363 of the semi-frustoconically-shaped portion 328. In other words, the semi-frustoconically-shaped portion 328 can be configured such that a first distance 362 between the apex 365 of the semi-frustoconically-shaped portion 328 and the first segment 342 is greater than a second distance 364 between a trough 363 of the semi-frustoconically-shaped portion 328 and the first segment 342. The apex 365 can be nearer the inner channel 327 than the trough 363. As a non-limiting example, the first distance 362 can be approximately 1 millimeter and the second distance 364 can be approximately 0.37 millimeters. As other non-limiting examples, the first distance 362 can be between 0.1 millimeters and 20 millimeters while the second distance 364 can be between approximately 0.1 millimeters and 19 millimeters.

The semi-frustoconically-shaped portion 328 can help to prevent the implant 300 from becoming twisted as it transitions to the deployed configuration (predetermined shape). For instance, by forming the semi-frustoconically-shaped portion 328 with an apex 365 disposed proximate the inner channel 327 and a trough 363 disposed distal from the inner channel 327, the semi-frustoconically-shaped portion 328 can prevent the formation of a sharp corner between the second segment 344 and the inner channel 327. By eliminating or reducing the sharp corner with the semi-frustoconically-shaped portion 328, the implant 300 is less likely to twist about the vertical axis Y as it transitions to the deployed configuration. Stated otherwise, the geometric shape formed by the semi-frustoconically-shaped portion 328 enables the implant 300 to resist twisting as it transitions to the deployed configuration.

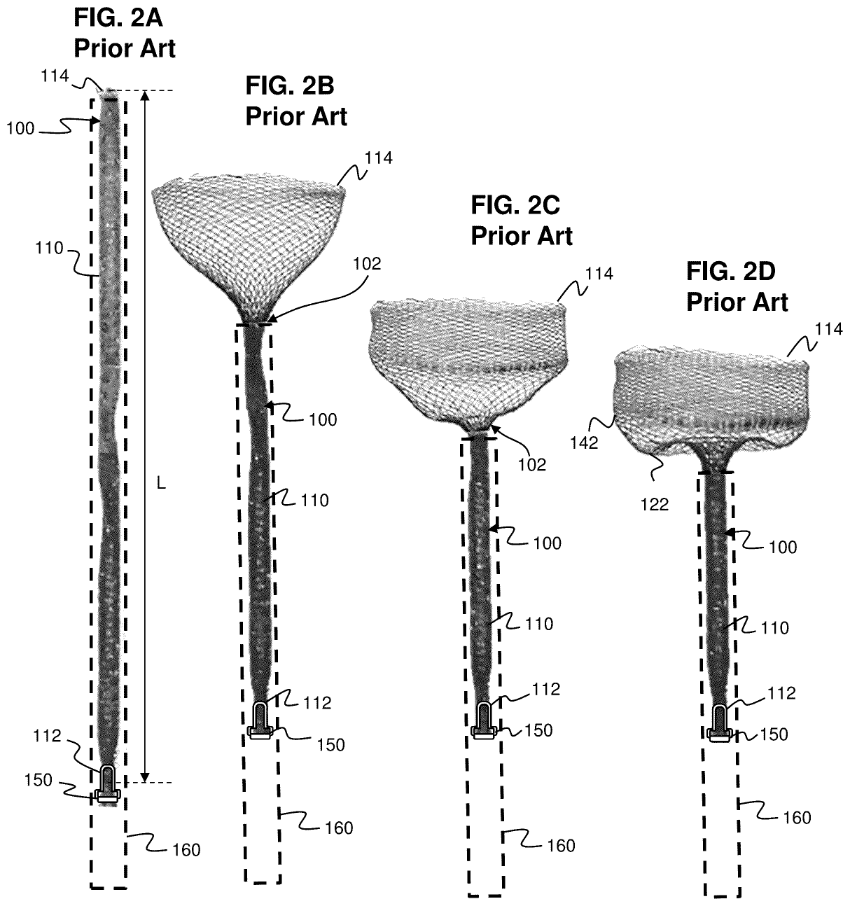

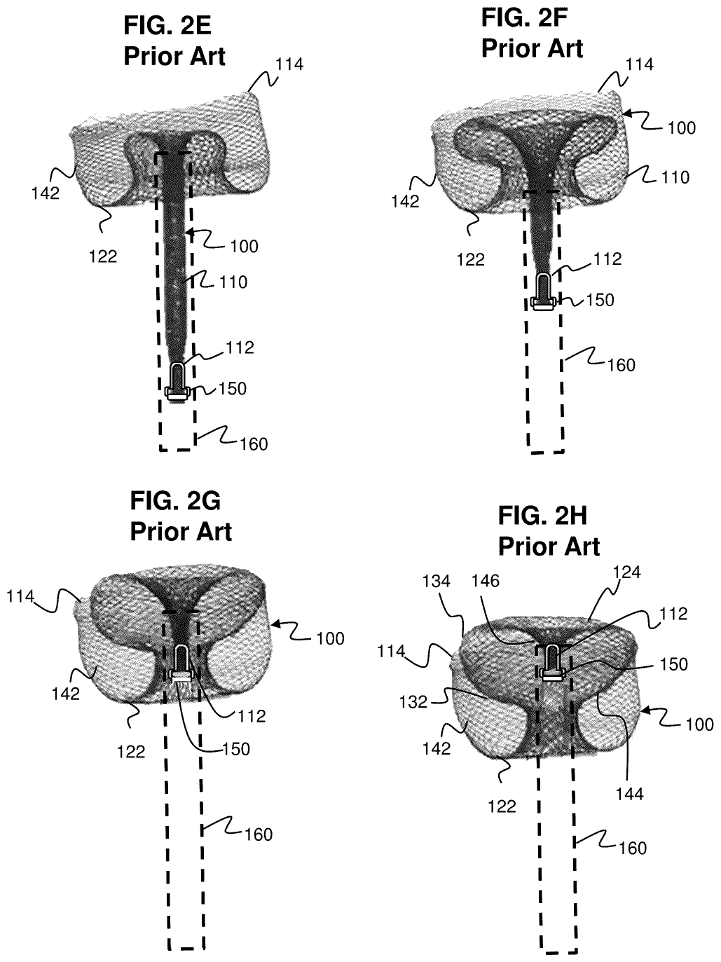

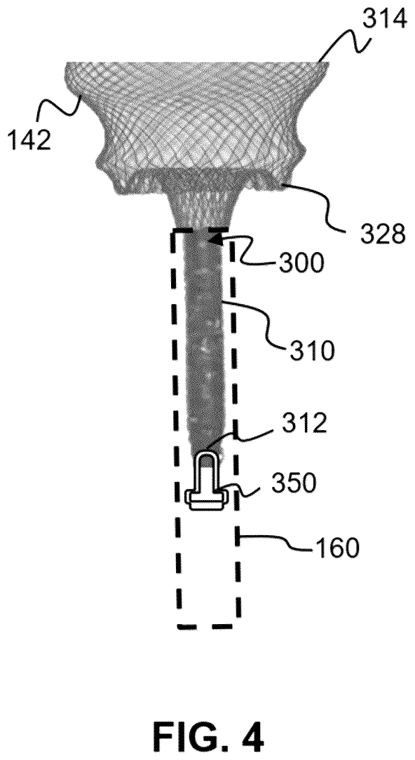

A further advantage of the semi-frustoconically-shaped portion 328 is that the semi-frustoconically-shaped portion 328 facilitates inversion of the braid 310 as it exits the microcatheter 160, making it easier to transition the implant 300 to the deployed configuration and reducing the likelihood that the implant 300 will become twisted. As illustrated in FIG. 4, if the implant 300 is pushed out of the microcatheter 160 and begins to transition to the predetermined shape (similar to the implant 100 as depicted in FIGS. 2C and 2D), the implant 300 begins to invert or turn inwardly because of the semi-frustoconically-shaped portion 328 of the implant 300. As previously described, it is generally at this point in the procedure that the existing implant 100 can become twisted at a twist point 202 (as illustrated in FIG. 2C). In contrast, by inverting or turning inwardly, as illustrated in FIG. 4, the implant 300 of the present disclosure facilitates an easier transition to the deployed configuration (predetermined shape) without requiring the physician to push on a delivery wire attached to detachment feature 350, reducing the likelihood that the implant 300 will twist and further increasing the likelihood that the implant 300 is installed properly.

Returning now to FIG. 3, the semi-frustoconically-shaped portion 328 can also help to prevent blood from flowing into the aneurysm when the implant 300 is in the deployed configuration and inserted in an aneurysm. To illustrate, the semi-frustoconically-shaped portion 328 can enable the implant 300 to include an inner channel 327 that is longer than an inner channel of comparable existing implants. The longer inner channel 327 can provide for added thrombogenicity. In other words, a longer inner channel 327 can include a greater amount of material and, therefore, can form a greater number of obstructions at which blood can form clots before reaching the inner braid 305. In this way, the implant 300 can be better able to prevent inflow of blood into the aneurysm when implanted and, therefore, can help promote healing of the aneurysm.

Similarly, it can be advantageous to minimize a neck opening 326 (reduce a diameter of the neck opening 326) defined by the semi-frustoconically-shaped portion 328 to maximize occlusion of an aneurysm neck when the implant 300 is implanted. The semi-frustoconically-shaped portion 328 can help reduce the neck opening 326 by pushing inwardly toward the central vertical axis Y when installed in an aneurysm. In this way, the semi-frustoconically-shaped portion 328 can further reduce inflow of blood into the aneurysm.

As another advantage, and as will be appreciated by one of skill in the art, by positioning the trough 363 proximate the first segment 342, the implant 300 can have a greater amount of material positioned (more layers of the implant 300) near an entrance of an aneurysm to help promote blood stasis and healing of the aneurysm. The semi-frustoconically-shaped portion 328 includes a curved portion extending between the apex 365 and the trough 363 to help position the trough 363 nearer the first segment 342 than existing implants 100. As will be appreciated by one of skill in the art, having the trough 363 of the semi-frustoconically-shaped portion 328 near the first segment 342 enables more material of the implant 300 to be positioned near an opening of the aneurysm, further facilitating clotting and blood flow diversion.

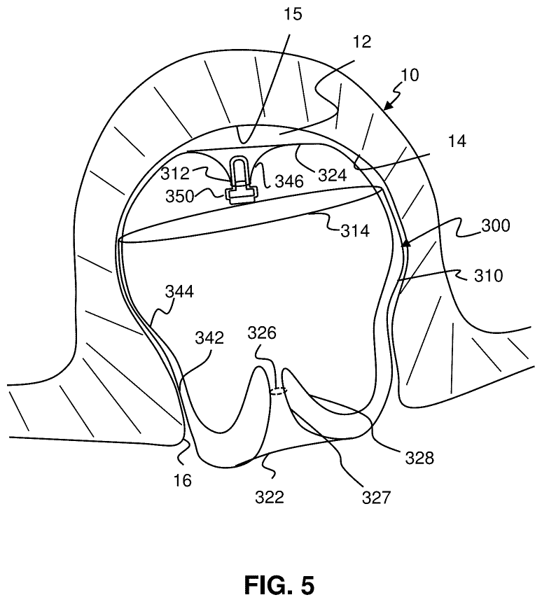

As illustrated in FIG. 5, when the implant 300 is installed into an aneurysm sac 12, the implant 300 can form an implanted shape. In the implanted shape, the braid 310 can have a first segment 342 contacting the aneurysm's wall 14, a second segment 344 nested within the first segment 342, a proximal inversion 322 positioned at the aneurysm's neck 16, and a distal inversion 324 positioned near a distal portion 15 of the aneurysm wall 14. In the implanted shape, the detachment feature 350 and pinched end 312 of the braid 310 can be suspended within the second segment 344.

As illustrated in FIG. 5, the tubular braid 310 in the implanted shape can be radially compressed and vertically extended compared to the predetermined shape. The first segment 342 in the implanted shape can correspond to the first segment 342 in the predetermined shape, the proximal inversion 322 in the implanted shape can correspond to the proximal inversion 322 adjacent to the first segment 342 in the predetermined shape, the second segment 344 in the implanted shape can correspond to the second segment 344 in the predetermined shape, the distal inversion 324 in the implanted shape can correspond to the distal inversion 324 adjacent to the third segment 346 in the predetermined shape, and a third segment 346 suspending the detachment feature 350 in the implanted shape can correspond to the third segment 346 in the predetermined shape. In the implanted shape, the second segment 344 can have a neck opening 326 corresponding to the neck opening 326 in the predetermined shape.

Depending on the shape of the aneurysm, when implanted in the implanted shape in aneurysms having a diameter that is significantly smaller than the aneurysm's height, the implanted shape can be radially compressed compared to the predetermined shape and the height of the braid in the implanted shape can be greater than the height of the braid in the predetermined shape.

As will be appreciated by one of skill in the art with the benefit of this disclosure, the semi-frustoconically-shaped portion 328 can help to reduce a diameter of the neck opening 326 and can form a comparatively longer inner channel 327 when in the implanted shape. For example, when in the implanted shape, the implant 300 can be compressed or collapsed inwardly to reduce a diameter of the neck opening 326, further promoting thrombogenicity. Furthermore, when in the implanted shape, the semi-frustoconically-shaped portion 328 can cause a greater amount of material to be positioned proximate the neck 16 of the aneurysm 10 to help further promote thrombogenicity as previously described.

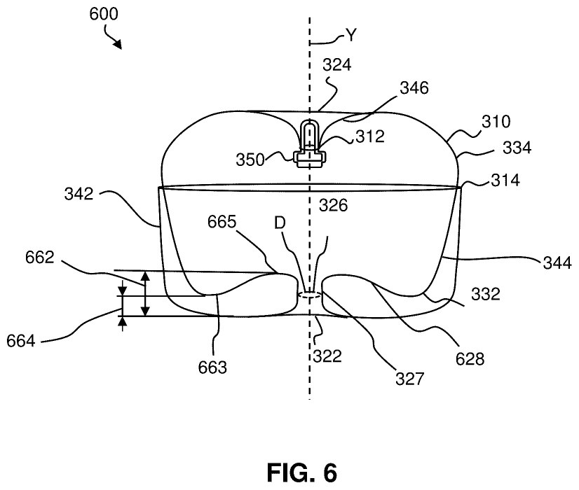

FIG. 6 illustrates another example implant 600 having a semi-frustoconically-shaped portion 628. The semi-frustoconically-shaped portion 628 can be similar to the semi-frustoconically-shaped portion 328 described herein except that the semi-frustoconically-shaped portion 628 can have a more rounded or smooth shape. In other words, semi-frustoconically-shaped portion 628 can have less of a slope between the apex 665 and the trough 663.

Similar to the semi-frustoconically-shaped portion 328, the first distance 662 between the apex 665 and the first segment 342 can be greater than a second distance 664 between the trough 663 and the first segment 342. The first distance 662 of implant 600 can be the same as, greater than, or less than the first distance 362 of implant 300. Similarly, the second distance 664 of implant 600 can be the same as, greater than, or less than the second distance 364 of implant 300. A distance between the trough 663 and the inner channel 327, however, can be greater than the distance between the trough 363 and the inner channel 327 of the implant 300. In this way, the semi-frustoconically-shaped portion 628 can have a greater outer diameter compared to the semi-frustoconically-shaped portion 328.

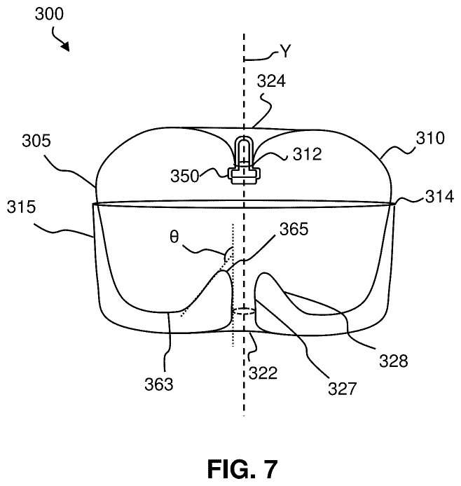

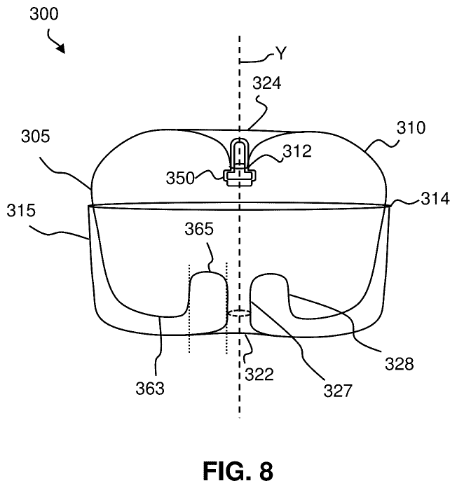

As illustrated in FIG. 7, the semi-frustoconically-shaped portion 328 can be formed such that the semi-frustoconically-shaped portion 328 extends outwardly at an angle from the apex 365 to the trough 363. The angle, for example, can be represented by the angle θ formed between a vertical line extending from the inner channel 327 and the length of the semi-frustoconically-shaped portion 328 extending between the apex 365 and the trough 363. As a non-limiting example, the angle θ can be approximately 100°, 115°, 130°, 145°, 160°, 180° (as illustrated in FIG. 8), or any other suitable angle for the application. As will be appreciated by one of skill in the art, the angle θ at which the semi-frustoconically-shaped portion 328 extends outwardly from the inner channel 327 can affect the distance at which the trough 363 is disposed from the inner channel 327 (i.e., a greater angle causing the trough 363 to be closer to the inner channel 327).

The example implants 300 and 600 described herein can rely on a radial outward force to anchor the implant within the sac of an aneurysm. To this end, the braid 310, 610 can be shaped to a predetermined shape having a diameter that is greater than its height so that the braid is radially constricted when implanted in an aneurysm. The ratio of diameter to height of the braid 310, 610 in a respective predetermined shape can be within the range of 2:1 to 1:3 to treat aneurysms of many known sizes and shapes.

In describing examples of the disclosed technology, terminology has been resorted to for the sake of clarity. As a result, not all possible combinations have been listed, and such variants are often apparent to those of skill in the art and are intended to be within the scope of the claims that follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

What is claimed is:

1. A system comprising:
a tubular braid comprising an open end, a pinched end, and a predetermined shape in which the tubular braid comprises a first segment extending from the open end to a first inversion, a second segment extending from the first inversion to a second inversion and forming a bag comprising a semi-frustoconically-shaped portion proximate the first inversion such that the semi-frustoconically-shaped portion defines an inner channel forming an opening to the bag, and a third segment surrounded by the second segment and extending from the second inversion to the pinched end; and
a catheter comprising a lumen therethrough, a distal end, and an outer diameter at the distal end being sized to be inserted into the bag through the opening of the bag.

2. The system of claim 1, wherein the semi-frustoconically-shaped portion is configured to prevent the tubular braid from becoming twisted proximate the inner channel when deployed.

3. The system of claim 1, wherein the semi-frustoconically-shaped portion is configured such that a first distance between an apex of the semi-frustoconically-shaped portion and the first segment is greater than a second distance between a trough of the semi-frustoconically-shaped portion and the first segment, the apex being nearer the inner channel than the trough.

4. The system of claim 3,
wherein the first distance is approximately 1 millimeter, and
wherein the second distance is approximately 0.37 millimeters.

5. The system of claim 3, wherein the semi-frustoconically-shaped portion forms an indentation into the bag proximate the opening.

6. The system of claim 3, wherein the inner channel extends from the first inversion to the apex of the semi-frustoconically-shaped portion.

7. The system of claim 1,
wherein the tubular braid is stable in an implanted shape based on the predetermined shape when constricted by a substantially spherical cavity, and
wherein, in the implanted shape, at least a portion of the first segment is positioned to contact a cavity wall of the substantially spherical cavity, a proximal inversion corresponding to the first inversion of the predetermined shape is positioned at an entrance to the substantially spherical cavity, the bag is positioned within the substantially spherical cavity, the opening of the bag is accessible at the entrance to the substantially spherical cavity, and the opening is configured to receive the distal end of the catheter into the bag.

8. The system of claim 7, wherein, in the implanted shape, the opening is resilient to expand to receive the distal end of the catheter and contract when the catheter is removed from the opening.

9. The system of claim 7, wherein, when in the implanted shape, the proximal inversion is configured such that the first segment forms an approximately flat surface proximate the entrance to the substantially spherical cavity.

10. The system of claim 7, wherein, in the predetermined shape, the tubular braid is cylindrically symmetrical about a central axis and the inner channel extends in a proximal direction from the bag, constricted about the central axis, and defining the opening of the bag.

11. The system of claim 10, wherein a diameter of the inner channel when the braid is in the predetermined shaped collapses when the braid is in the implanted shape.

12. The system of claim 10, wherein the inner channel is sized to facilitate clotting of blood when the braid is in the implanted shape.

13. The system of claim 1, wherein an outer profile of the tubular braid in the predetermined shape is approximately a right cylinder.

14. The system of claim 1, wherein, in the predetermined shape, the open end encircles the bag.

15. The system of claim 1, wherein the tubular braid comprises a shape memory material configured to self-expand into the predetermined shape.

16. The system of claim 15, wherein the tubular braid comprises at least one of nitinol and platinum.

17. A tubular braid comprising:
an open end;
a pinched end; and
a predetermined shape in which the tubular braid comprises:
a first segment extending from the open end to a first inversion;
a second segment extending from the first inversion to a second inversion and forming a bag comprising a semi-frustoconically-shaped portion proximate the first inversion such that the semi-frustoconically-shaped portion defines an inner channel forming an opening to the bag; and
a third segment surrounded by the second segment and extending from the second inversion to the pinched end.

18. The tubular braid of claim 17, wherein the semi-frustoconically-shaped portion is configured to prevent the tubular braid from becoming twisted proximate the inner channel when deployed.

19. The tubular braid of claim 17, wherein the semi-frustoconically-shaped portion is configured such that a first distance between an apex of the semi-frustoconically-shaped portion and the first segment is greater than a second distance between a trough of the semi-frustoconically-shaped portion and the first segment, the apex being nearer the inner channel than the trough.

20. The tubular braid of claim 19,
wherein the first distance is approximately 1 millimeter, and
wherein the second distance is approximately 0.37 millimeters.
